(19) United States
(12) Patent Application Publication
(10) Pub. No.: US 2005/0260131 A1
(43) Pub. Date: Nov. 24, 2005

(54) Pharmaceuticals for enhanced delivery to disease targets

(75) Inventors: Mohan Mark Amaratunga, Clifton Park, NY (US); Bahram Moasser, Schenectady, NY (US); Faisal Ahmed Syud, Clifton Park, NY (US); John Bucknam Brogan, Niskayuna, NY (US); Daniel Joshua Kramer, Clifton Park, NY (US)

Correspondence Address: GENERAL ELECTRIC COMPANY;GLOBAL RESEARCH, PATENT DOCKET RM. BLDG. K1-4A59, NISKAYUNA, NY, 12309, US

(73) Assignee: General Electric Company

(21) Appl. No.: 10/851,359

(22) Filed: May 20, 2004

(51) Int. Cl. A61K 51/00; A61K 49/00; C07K 14/52; C07K 16/46; A61K 48/00

(52) U.S. Cl. 424/1.49; 424/9.34; 424/85.1; 530/351; 530/391.1; 530/400; 536/23.1; 514/44

(57) ABSTRACT

Pharmaceuticals for enhanced delivery to a disease target comprises a pair of compounds. The first compound comprises a first oligopeptide conjugated to a first moiety for coupling with a diagnostic or therapeutic active agent. The second compound comprises a second oligopeptide conjugated to a targeting species having a targeting moiety capable of binding to a target. The second oligopeptide has a sequence that is complementary to a sequence of the first oligopeptide. The first and second oligopeptides can be complementary PNA sequences. The pharmaceuticals are administered into a subject in methods for diagnosing or treating a disease condition, or assessing the effectiveness of a treatment of the disease condition.

### BACKGROUND OF THE INVENTION

The present invention relates to pharmaceuticals for enhanced delivery to disease targets. In particular, the present invention relates to such pharmaceuticals for enhanced delivery of diagnostic or therapeutic agents to disease sites based on the pretargeting strategy.

The growing need for the early diagnosis and assessment and/or treatment of disease can potentially be addressed by pharmaceuticals that preferentially accumulate at the disease sites. In diagnostic applications, these pharmaceuticals can elucidate the state of the disease through its distinctive molecular biology expressed as disease markers that are not present, or are present in diminished levels, in healthy tissues. In therapeutic applications, these pharmaceuticals can deliver an enhanced dose of therapeutic agents to the disease sites through specific interactions with the disease markers. By specifically targeting physiological or cellular functions that are present only in disease states, these pharmaceuticals can report exclusively on the scope and progress of that disease or exclusively target the diseased tissue. A signal-generating moiety is a key element of these diagnostic pharmaceuticals, which produce differentiated signals at the disease sites.

The detection of a target site benefits from a high signal-to-background ratio of detection agent. Therapy benefits from as high an absolute accretion of therapeutic agent at the target site as possible, as well as a reasonably long duration of binding. In order to improve the targeting ratio and amount of agent delivered to a target site, the use of targeting vectors comprising diagnostic or therapeutic agents conjugated to a targeting moiety for preferential localization has long been known.

Examples of targeting vectors include diagnostic or therapeutic agent conjugates of targeting moieties such as antibody or antibody fragments, cell- or tissue-specific peptides, and hormones and other receptor-binding molecules. For example, antibodies against different determinants associated with pathological and normal cells, as well as associated with pathogenic microorganisms, have been used for the detection and treatment of a wide variety of pathological conditions or lesions. In these methods, the targeting antibody is directly conjugated to an appropriate detecting or therapeutic agent.

One problem encountered in direct targeting methods, i.e., in methods wherein the diagnostic or therapeutic agent (the “active agent”) is conjugated directly to the targeting moiety, is that a relatively small fraction of the conjugate actually binds to the target site, while the majority of conjugate remains in circulation and compromises in one way or another the function of the targeted conjugate (i.e., the conjugate accumulated or bound at the target). In the case of a diagnostic conjugate (e.g., a radioimmunoscintigraphic or magnetic resonance imaging conjugate), non-targeted conjugate, which remains in circulation, can increase background and decrease resolution. In the case of a therapeutic conjugate having a very toxic therapeutic agent (e.g., a radioisotope, drug, or toxin) attached to a long-circulating targeting moiety such as an antibody, circulating conjugate can result in unacceptable toxicity to the host, such as marrow toxicity or systemic side effects.

Pretargeting methods have been developed to increase the target-to-background ratios of the detection or therapeutic agents. In pretargeting methods, a primary targeting species (which is not bound to a diagnostic or therapeutic agent) is targeted to an in vivo target site. The primary targeting species comprises a first targeting moiety, which binds to the target site, and a second moiety, which presents a binding site available for binding by a subsequently administered second targeting species. Once sufficient accretion of the primary targeting species is achieved, the second targeting species comprising a diagnostic or therapeutic agent and a second targeting moiety, which recognizes the available binding site of the primary targeting species, is administered.

A favorite pretargeting approach has used the well-known mutual binding property of the biotin/avidin (or streptavidin) pair. For example, this approach has been used to administer a cytotoxic radioantibody to a tumor. In a typical procedure, a monoclonal antibody targeted against a tumor-associated antigen is conjugated to avidin (or biotin) and administered to a patient who has a tumor recognized by the antibody. Then the therapeutic agent, e.g., a chelated radionuclide covalently bound to biotin (or avidin), is administered. The radionuclide, via its attached biotin (or avidin), is taken up by the antibody-avidin (or -biotin) conjugate pretargeted to the tumor.

Pretargeting strategy offers certain advantages over the use of direct targeting methods. For example, use of the pretargeting strategy for the in vivo delivery of radionuclides to a target for therapy, e.g., radioimmunotherapy, reduces the marrow toxicity caused by prolonged circulation of a radioimmunoconjugate. This is because the radioisotope is delivered as a rapidly clearing, low molecular weight chelate rather than directly conjugated to a primary targeting molecule, which is often a long-circulating species.

Despite these advantages, pretargeting strategy, as it has been practiced to date, suffers from certain drawbacks. First among these is the very low amount of active agent delivered to the target site compared to when the active agent is directly attached to an antibody, for a variety of reasons. Second, the active agent-carrying vectors, which are often peptides, are often degraded by endogenous proteases in the body. In particular, radiolabeled biotins may be subject to plasma biotindase degradation. Furthermore, when conjugated to antibodies, streptavidin and avidin can generate anti-streptavidin or anti-avidin antibodies in a patient. In addition, the potential effects of endogenous biotin during in vivo pretargeting can lead to the disappearance of biotin binding expression because of saturation by biotin.

A need exists, therefore, for improved diagnostic and therapeutic pharmaceuticals for use with the pretargeting strategy. It is very desirable to provide such pharmaceuticals that have fewer side effects to a patient than those exhibited in the prior-art pharmaceuticals in this area.

### SUMMARY OF THE INVENTION

Diagnostic or therapeutic compounds or pharmaceuticals designed for use in a pretargeting strategy comprise a first oligopeptide that is conjugated to a first moiety for coupling with a diagnostic or therapeutic active agent. The first oligopeptide is capable of binding to a second oligopeptide that comprises a sequence complementary to the first oligopeptide. The second oligopeptide is conjugated to a targeting species having a targeting moiety that is capable of binding to a target. The target is sometimes herein referred to as a marker substance or a biomarker.

According to one aspect of the present invention, the first oligopeptide is a peptide nucleic acid (“PNA”) that has a backbone chain comprising repeating units of N-(2-amino-ethyl)-glycine, wherein the amino nitrogen of the glycine moiety is linked to one of four heterocyclic bases through a methyl carbonyl linkage. The four heterocyclic bases are adenine (conventionally abbreviated as “A”), guanine (conventionally abbreviated as “G”), cytosine (conventionally abbreviated as “C”), and thymine (conventionally abbreviated as “T”), which are normally found in deoxyribonucleic acid (“DNA”) sequences. In the PNAs of the present invention, some or all of the thymine moieties may be substituted by uracil (conventionally abbreviated as “U”). Thus, the repeating units of a PNA of the present invention has a formula of: 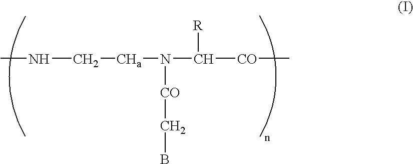
wherein B is one of four heterocyclic bases A, G, C, and T (or U); and R is selected from the group consisting of the side groups covalently bonded to the α-carbons of the twenty known α-amino acids (i.e., glycine, alanine, valine, leucine, isoleucine, serine, cysteine, threonine, methionine, praline, phenylalanine, tyrosine, tryptophan, histidine, lysine, arginine, aspartic acid, glutamic acid, asparagines, glutamine). Adenine and guanine are attached to the —CO—CH₂— linkage at nitrogen 9, and cytosine and thymine at nitrogen 1. It should be understood that the terminal groups of the PNA sequence in solution are NH₃⁺ and COO⁻.

According to another aspect of the present invention, n is an integer in the range from about 4 to about 20, preferably from about 6 to about 14, and more preferably from about 8 to about 12.

According to still another aspect of the present invention, the targeting moiety is selected from the group consisting of proteins, peptides, polypeptides, glycoproteins, lipoproteins, phospholipids, oligonucleotides, steroids, alkaloids or the like, e.g., hormones, lymphokines, growth factors, albumin, cytokines, enzymes, immune modulators, receptor proteins, antisense oligonucleotides, antibodies and antibody fragments, which preferentially bind biomarkers that are produced by or associated with the target site.

Other features and advantages of the present invention will be apparent from a perusal of the following detailed description of the invention and the accompanying drawings in which the same numerals refer to like elements.

### BRIEF DESCRIPTION OF THE DRAWINGS

FIG. 1 shows schematically a pair of active agent-labeled species and pretargeting conjugate of the present invention for MRI application.

FIG. 2 shows schematically a pair of radioactive-labeled species and pretargeting conjugate of the present invention for PET application.

### DETAILED DESCRIPTION OF THE INVENTION

The following terms are used in the present disclosure:

Target site: A specific site to which a diagnostic or therapeutic agent is to be delivered, such as a cell or group of cells, tissue, organ, tumor, or lesion.

Targeting moiety: A moiety that binds to the target site or to a substance produced by or associated with the target site via a primary binding site. Non-limiting examples of such a moiety are proteins, peptides, polypeptides, glycoproteins, lipoproteins, phospholipids, oligonucleotides, steroids, alkaloids or the like, e.g., hormones, lymphokines, growth factors, albumin, cytokines, enzymes, immune modulators, receptor proteins, antisense oligonucleotides, antibodies and antibody fragments, which preferentially bind marker substances that are produced by or associated with the target site.

Complementary PNA sequence: A PNA sequence that binds to another PNA sequence by base pairing.

The present invention provides diagnostic or therapeutic compounds or pharmaceuticals designed for use in a pretargeting strategy. For each application, the present invention provides two species: a first and a second species. The first species (also herein referred to as “active agent-labeled species”) comprises a first oligopeptide that is conjugated to a first moiety for coupling with a diagnostic or therapeutic active agent. The first oligopeptide is capable of binding to a second oligopeptide that comprises a sequence complementary to the first oligopeptide. The second species (also herein referred to as “pretargeting conjugate”) comprises the second oligopeptide that is conjugated to a targeting species having a targeting moiety that is capable of binding to a target site or to a substance produced by or associated with the target site.

In one aspect, the present invention provides a kit that comprises the active agent-labeled species and the pretargeting conjugate kept separately before use for purposes of diagnosing or treating diseases.

According to one aspect of present invention, the first oligopeptide is a first PNA sequence, the backbone chain of which comprises repeating units of substituted N-(2-amino-ethyl)-glycine residues, as described above. The second oligopeptide comprises a second PNA sequence that is complementary to the first PNA sequence. The second PNA sequence binds to the first PNA sequence by base pairing; i.e., A forms hydrogen bonding with T (or U), and G with C.

According to an embodiment of the present invention, the targeting moiety comprises an antibody or an antibody fragment that preferentially binds biomarkers that are produced by or associated with the target site.

Diagnostic and Therapeutic Active Agents

Among the diagnostic and therapeutic active agents applicable to and useful in the present invention, gamma-emitters, positron-emitters, x-ray emitter, paramagnetic ions and fluorescence-emitters are suitable for detection and/or therapy, while beta- and alpha-emitters and neutron-capturing agents, such as boron and uranium, also can be used for therapy.

Suitable radioisotopes for coupling with the first oligopeptide to produce diagnostic or therapeutic active agents and used in diagnostic or therapeutic methods of the present invention include: actinium-225, astatine-211, iodine-120, iodine-123, iodine-124, iodine-125, iodine-126, iodine-131, iodine-133, bismuth-212, arsenic-72, bromine-75, bromine-76, bromine-77, indium-110, indium-111, indium-113m, gallium-67, gallium-68, strontium-83, zirconium-89, ruthenium-95, ruthenium-97, ruthenium-103, ruthenium-105, mercury-107, mercury-203, rhenium-186, rhenium-188, tellurium-121m, tellurium-122m, tellurium-125m, thulium-165, thulium-167, thulium-168, technetium-94m, technetium-99m, fluorine-18, silver-111, platinum-197, palladium-109, copper-62, copper-64, copper-67, phosphorus-32, phosphorus-33, yttrium-86, yttrium-90, scandium-47, samarium-153, lutetium-177, rhodium-105, praseodymium-142, praseodymium-143, terbium-161, holmium-166, gold-199, cobalt-57, cobalt-58, chromium-51, iron-59, selenium-75, thallium-201, and ytterbium-169. Preferably the radioisotope will emit a particle or ray in the 10-7,000 keV range, more preferably 50-1,500 keV.

Isotopes preferred for imaging applications include: iodine-123, iodine-125, iodine-131, indium-111, gallium-67, ruthenium-97, technetium-99m, cobalt-57, cobalt-58, chromium-51, iron-59, selenium-75, thallium-201, ytterbium-169, copper-64, and fluorine-18.

Isotopes preferred for therapeutic use include: actinium-225, bismuth-212, lead-212, bismuth-213, iodine-125, iodine-131, rhenium-186, rhenium-188, silver-11, platinum-197, palladium-109, copper-67, copper-64, phosphorus-32, phosphorus-33, yttrium-90, scandium-47, samarium-153, lutetium-177, rhodium-105, praseodymium-142, praseodymium-143, terbium-161, holmium-166, and gold-199.

In one aspect of the present invention, the diagnostic active agent is a magnetic resonance imaging contrast agent, which serves to enhance the contrast of images obtained in magnetic resonance imaging procedure. Suitable paramagnetic ions that are useful for magnetic resonance imaging (“MRI”) are those of elements having atomic numbers of 21-29, 42, 44, and 58-70. Particularly useful are gadolinium ion and iron metal, ion, or oxides. Preferably, gadolinium ions are bound by chelators, such as polycarboxylic acids (carboxylic acids having a plurality of —COOH groups), which are conjugated directly or indirectly to the first oligopeptide through one of the —CO(O)— groups. Non-limiting examples of such chelators are diethylenetriamine-pentaacetic acid (“DTPA”); 1,4,7,10-tetraazacyclododecane-N,N′,N″,N′″-tetraacetic acid (“DOTA”); p-isothiocyanatobenzyl-1,4,7,10-tetraazacyclododecane-1,4,7,10-tetraacetic acid (“p-SCN-Bz-DOTA”); 1,4,7,10-tetraazacyclododecane-N,N′,N″-triacetic acid (“DO3A”); 1,4,7,10-tetraazacyclododecane-1,4,7,10-tetrakis(2-propionic acid) (“DOTMA”); 3,6,9-triaza-12-oxa-3,6,9-tricarboxymethylene-10-carboxy-13-phenyl-tridecanoic acid (“B-19036”); 1,4,7-triazacyclononane-N,N′,N″-triacetic acid (“NOTA”); 1,4,8,11-tetraazacyclotetradecane-N,N′,N″,N′″-tetraacetic acid (“TETA”); triethylene tetraamine hexaacetic acid (“TTHA”); trans-1,2-diaminohexane tetraacetic acid (“CYDTA”); 1,4,7,10-tetraazacyclododecane-1-(2-hydroxypropyl)4,7,10-triacetic acid (“HP-DO3A”); trans-cyclohexane-diamine tetraacetic acid (“CDTA”); trans(1,2)-cyclohexane diethylene triamine pentaacetic acid (“CDTPA”); 1-oxa-4,7,10-triazacyclododecane-N,N′,N″-triacetic acid (“OTTA”); 1,4,7,10-tetraazacyclododecane-1,4,7,10-tetrakis{3-(4-carboxyl)-butanoic acid}; 1,4,7,10-tetraazacyclododecane-1,4,7,10-tetrakis(acetic acid-methyl amide); 1,4,7,10-tetraazacyclododecane-1,4,7,10-tetrakis(methylene phosphonic acid); and derivatives thereof.

Superparamagnetic metal oxides, such as iron, chromium, cobalt, manganese, nickel, and tungsten oxide, are also suitable for generating magnetic resonance signal useful for diagnostic purposes, as disclosed, for example, in U.S. Pat. Nos. 5,492,814 and 5,314,679, which are incorporated in their entirety herein by reference. Such metal oxides are preferably present in nanometer-sized aggregates (e.g. from about 10 nm to about 500 nm), either uncoated or preferably coated with a shell comprising a biologically compatible material, such as polysaccharide, poly(amino acid), or organosilane. The coated or uncoated metal oxide aggregates are then covalently attached directly or indirectly (through a linker) to the first oligopeptide to produce an MRI signal generating species. Preferably, the first oligopeptide is a PNA, and the attachment is effected at the terminal amino or carboxylic group of the PNA sequence. Methods for attachment of metal oxide (such as iron oxide) particles to organic materials, such as proteins, are known and disclosed, for example, in U.S. Pat. Nos. 5,492,814 and 4,628,037, which are incorporated herein by reference.

An MRI Active Agent-Labeled Species

In a preferred embodiment, a moiety that is capable of providing an effective magnetic resonance signal and that can serve as a contrast-enhancing agent for MRI comprises an extended poly(amino acid) homopolymer or copolymer. A plurality of amino acid residues of the poly(amino acid) is conjugated to chelators that form coordination complexes with paramagnetic ions. For example, suitable chelators for this embodiment of the present invention are polycarboxylic acids and are disclosed herein above. Suitable poly(amino acid) homopolymers and copolymers are polylysine, polyhistidine, polyarginine, polyasparagine, polyglutamine, poly(glutamic acid), poly(aspartic acid), and copolymers of at least two amino acids selected from the group consisting of lysine, histidine, arginine, asparagine, glutamine, glutamic acid, and aspartic acid. Methods of producing MRI contrast enhancing agents based on these poly(amino acid) conjugates are described in U.S. Pat. Nos. 5,762,909; 6,537,521; 6,685,915; and U.S. patent application Ser. Nos. 10/609,269 and 10/638,888, which have the common assignee and are incorporated herein in their entirety by reference. In a preferred embodiment, the poly(amino acid) conjugate is polylysine, polyglutamic acid, or copolymer of glutamic acid and lysine, wherein ninety percent or more (the degree of conjugation to chelators) of the lysine residues are conjugated to diethylenetriamine pentaacetic acid chelator, each of which forms a coordination complex with a gadolinium ion. In another embodiment, the chelator is DOTA. Preferably, the degree of conjugation to chelators is at least 95 percent, and more preferably at least 98 percent. The poly(amino acid) conjugate is then linked or otherwise attached to the first oligopeptide, such as the first PNA sequence, by an amide bond formed by the reaction of the free terminal amino group of the poly(amino acid) conjugate and the free terminal carboxylic acid group of the first PNA sequence (or the free terminal carboxylic acid group of the poly(amino acid) conjugate and the free terminal amino group of the first PNA sequence). The formation of this amide bond is a conventional reaction and is well known. Thus, in a preferred embodiment, the active agent-labeled species has the formula:
{E_{q}Jᵣ}ₘ-D-A (II)
wherein A is 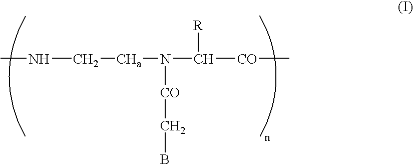
D is a direct bond or a linker having the formula (—CH₂—CH₂—O—)ₚ; 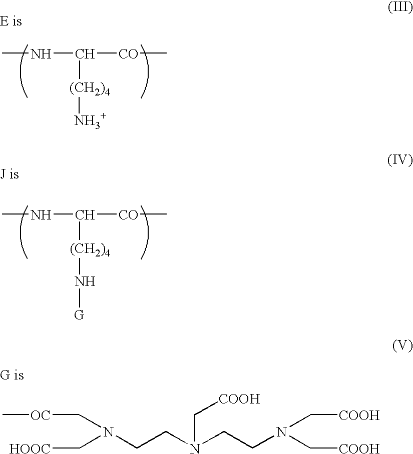
n is disclosed above;
p is from about 1 to about 50, preferably from about 1 to 20, more preferably from about 1 to about 10;
q is 0 or 1;
r is 0 or 1; at least one of q and r is non-zero; and
m is from about 10 to about 600; provided that the ratio of r/(q+r) is from about 0.9 to about 0.98. It should be understood that, in formula (II), there are m units of E_{q}Jᵣ, wherein q and r of each one of the units take on values independent from those of other units. In other words, the repeating residues E and J in the polymer species represented by {E_{q}Jᵣ}ₘ are not necessarily connected in any particular order. The chelator G then serves to form a complex with a paramagnetic ion, such as gadolinium or dysprosium. Preferably, the paramagnetic ion is gadolinium.

Although E and J in formula (II) are shown to be residues of lysine, they may be chosen independently from the list of other amino acid residues disclosed above to form the desired poly(amino acid) represented by {E_{q}Jᵣ}ₘ. In addition, G may be chosen from among the chelators disclosed above.

PET or SPECT (Single Photon Emission Computed Tomography) Active Agent-Labeled Species

In another preferred embodiment of the present invention, a moiety that is capable of providing an effective positron emission tomography (“PET”) signal is linked, directly or indirectly, to the first oligopeptide, such as a PNA sequence.

In one preferred embodiment, a moiety containing a radioisotope of iodine (such as I-123, I-124, or I-125) or F-18 is conjugated to the first oligopeptide (e.g., a PNA sequence) to provide the PET active agent-labeled species. The first oligopeptide (or PNA) is provided with a lysine moiety among the residues of amino acids of the oligopeptide chain, preferably at one of its termini. In one embodiment, 4-iodobenzamide is linked directly to the free amino group on the terminal lysine moiety of the first oligopeptide (or PNA). In another embodiment, 4-iodobenzamide is linked to the free amino group of the terminal lysine moiety of a short peptide linker comprising about 2 to about 20 amino acid residues. The opposite terminal of the short peptide linker is then linked to the free amino group of the terminal lysine residue on the first oligopeptide (or PNA). The iodine atom in the 4-iodobenzamide is chosen to provide the desired radioisotope. In another embodiment, 4-fluorobenzamide is linked to the free amino group on the terminal lysine moiety of the first oligopeptide (or PNA), wherein the fluorine atom is radioisotope of F-18. The synthesis of an 18-mer oligopeptide conjugated to 4-iodobenzamide is as follows:

Solid phase synthesis of the 18-mer peptide was performed on a Rainin Symphony peptide synthesizer employing the Fmoc (9-fluorenyl-methoxycarbonyl) method. The synthesis was conducted on a 25 Fmole scale and the standard coupling protocol required 125 μmole of Fmoc-protected amino acids activated by 2-(1H-benzotriazole-1-yl)-1,1,3,3-tetramethyluronium hexafluorophosphate (HBTU). The Fmoc-protected amino acids were purchased from Advanced Chemtech (Louisville, Ky.). The synthesis was conducted specifically to have a lysine moiety at one end of the 18-mer peptide. Peptides and peptide conjugates were purified by reverse phase HPLC using a Rainin HPXL system and a Vydac C4 protein column using gradients of A:0.1% aqueous TFA, B:0.1% TFA in acetonitrile (gradient from 95% A/5% B to 50% A/50% B over 30 minutes) with detection at 220 nm. Mass spectroscopy was conducted using an Applied Biosystems Voyager instrument.

Following final removal of the N-terminal Fmoc group, the resin was suspended in Ac₂O and HBTU in DMF for 30 minutes. After completing the standard rinsing sequence the peptide was cleaved in 95% aqueous TFA with 2.5% triisopropylsilane and precipitated with diethyl ether. The precipitate was washed with diethyl ether twice and then extracted between ether and water. The aqueous phase was freeze dried to provide a solid. Purification by HPLC provided the title compound. MALDI MS found 1712.

A stirred solution of the 18-mer peptide in DMSO was treated with 4-iodobenzoic acid NHS ester in about 1 ml 0.4 M N-methyl morpholine/DMF and about 1 ml DMSO for 4.5 hours. The solution was diluted with water and concentrated by freeze drying. MALDI MS found 1963.

It should be recognized that this synthesis protocol is applicable to any desired peptide sequence of any desired length. Thus, the iodobenzamide-conjugated 18-mer peptide may be represented by the following formula:

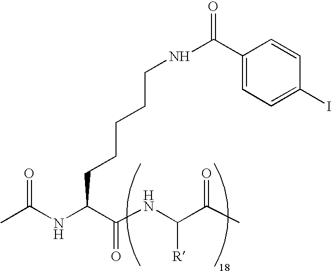

In this formula, R′ is independently selected from the group consisting of the side groups covalently bonded to the α-carbons of the twenty known α-amino acids, and these side groups having substitutions, for example, with heteroatoms. In other words, each of the eighteen amino acids may be independently selected. It should be understood that any number of amino acid residues other than 18 is also possible.

This 18-mer peptide may serve as a linker to the first oligopeptide (or PNA). In addition, the 18-mer peptide in this example can be replaced by the first oligopeptide (or PNA) itself. In that case, the oligopeptide (or PNA) is labeled directly with an iodinated PET active agent.

In another example, the 18-mer peptide was linked to a 1,4,8,11-tetraazacyclotetradecane-N,N′,N″,N′″-tetraacetic acid (“TETA”) moiety, which serves as the chelator for PET radioisotopic metals, such as Cu-64 or Cu-67. The conjugation of the TETA moiety to the 18-mer peptide is as follows:

A sample of the 18-Mer peptide was prepared according to the protocol described above. In this case the lysine residue was protected at the epsilon position with the acid sensitive Mtt (methoxy trityl) group. Prior to removal of the terminal Fmoc group, the resin was treated with 3% TFA in methylene chloride (3×) to remove the Mtt group. Following an extensive washing sequence, the resin was suspended in 0.4 M N-methyl morpholine/DMF and treated with tri tert-butyl TETA and 2-(1H-azabenzotriazole-1-yl)-1,1,3,3-tetramethyluronium hexafluorophosphate (HATU). The tri tert-butyl TETA was synthesized by modification of the published procedure (J. L. Lewis et al., J. Med. Chem., Vol. 42, 1341-47 (1999). Following standard Fmoc cleavage and acetylation with HBTU and Ac₂O in DMF for 30 minutes, the peptide was cleaved as described above to provide the title compound as a mixture with the starting peptide. MALDI MS found 2125. The TETA-conjugated 18-mer peptide has the following formula: 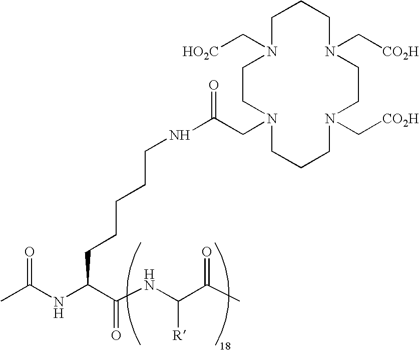
wherein R′ is independently selected from the group consisting of the side groups covalently bonded to the α-carbons of the twenty known α-amino acids, and these side groups having substitutions, for example, with heteroatoms. In other words, each of the eighteen amino acids may be independently selected. It should be understood that any number of amino acid residues other than 18 is also possible.

This 18-mer peptide may serve as a linker to the first oligopeptide (or PNA). Alternatively, the 18-mer peptide in this example can be replaced by the first oligopeptide (or PNA) itself. In that case, the oligopeptide (or PNA) is labeled directly with a PET or SPECT active agent, which comprises a radioisotopic metal ion bound by a chelator.

As yet another alternative, the active agent-labeled species comprises a poly(amino acid) chain (e.g., polylysine), wherein at least ninety percent of the free amino side groups are conjugated to a carboxylic acid having a plurality of carboxylic groups, and wherein one or more of the remaining free amino side groups are conjugated to a PET or SPECT signal-generating moiety; e.g., 4-iodobenzamide, 4-fluorobenzamide, or a chelating moiety that forms a coordination complex with a PET or SPECT signal-generating metal ion. The chelating moiety can be TETA or one of the carboxylic acids having a plurality of carboxylic groups, as disclosed above.

Therapeutic Agents

Among the therapeutic agents useful in the current invention are isotopes, drugs, toxins, fluorescent dyes activated by nonionizing radiation, hormones, hormone antagonists, receptor antagonists, enzymes or proenzymes activated by another agent, autocrine, or cytokine. Many drugs and toxins are known which have cytotoxic effects on cells. They can be found in compendia of drugs and toxins, such as the Merck Index, Goodman and Gilman's “The Pharmacological Basis of Therapeutics” (Tenth Edition, McGraw-Hill, New York, 2001), and the like, and in the references cited in U.S. patents incorporated herein by reference. Any such drug can be conjugated, coupled, attached to, or loaded onto peptides, proteins, polymers, or PNAs of the present invention by conventional means and/or chemistry well known in the art. Specific embodiments of the present invention of such conjugation, coupling, attachment, or loading are disclosed herein below.

The present invention also contemplates dyes used, for example, in photodynamic therapy, conjugated to peptides, proteins, polymers, or PNAs of the present invention used in conjunction with appropriate nonionizing radiation.

The use of light and porphyrins in methods of the present invention is also contemplated and their use in cancer therapy has been reviewed by van den Bergh (Chemistry in Britain, May 1986, Vol. 22, pp. 430-437), which is incorporated herein in its entirety by reference.

Examples of known cytotoxic agents useful in the present invention are listed in Goodman and Gilman's “The Pharmacological Basis of Therapeutics,” Tenth Edition, McGraw-Hill, New York, 2001. These include taxol; nitrogen mustards, such as mechlorethamine, cyclophosphamide, melphalan, uracil mustard and chlorambucil; ethylenimine derivatives, such as thiotepa; alkyl sulfonates, such as busulfan; nitrosoureas, such as carmustine, lomustine, semustine and streptozocin; triazenes, such as dacarbazine; folic acid analogs, such as methotrexate; pyrimidine analogs, such as fluorouracil, cytarabine and azaribine; purine analogs, such as mercaptopurine and thioguanine; vinca alkaloids, such as vinblastine and vincristine; antibiotics, such as dactinomycin, daunorubicin, doxorubicin, bleomycin, mithramycin and mitomycin; enzymes, such as L-asparaginase; platinum coordination complexes, such as cisplatin; substituted urea, such as hydroxyurea; methyl hydrazine derivatives, such as procarbazine; adrenocortical suppressants, such as mitotane; hormones and antagonists, such as adrenocortisteroids (prednisone), progestins (hydroxyprogesterone caproate, medroprogesterone acetate and megestrol acetate), estrogens (diethylstilbestrol and ethinyl estradiol), antiestrogens (tamoxifen), and androgens (testosterone propionate and fluoxymesterone).

Drugs that interfere with intracellular protein synthesis can also be coupled to the first oligopeptide of the present invention; such drugs are known to these skilled in the art and include puromycin, cycloheximide, and ribonuclease.

Toxins can also be coupled to the first oligopeptide of the present invention. Toxins useful as therapeutics are known to those skilled in the art and include plant and bacterial toxins, such as, abrin, alpha toxin, diphtheria toxin, exotoxin, gelonin, pokeweed antiviral protein, ricin, and saporin.

Toxins in their native form require a minimum of three different biochemical functions to kill cells: a cell binding function, a cytotoxic function, and a function to translocate the toxic activity into the cells.

Other therapeutic agents useful in the present invention include anti-DNA, anti-RNA, radiolabeled oligonucleotides, such as anti-sense oligodeoxyribonucleotides, anti-protein and anti-chromatin cytotoxic, or antimicrobial agents.

Targeting Species

The targeting species that is conjugated to the second oligopeptide to form the pretargeting conjugate can be a compound or a fragment of a compound. The targeting species has a targeting moiety that binds to a target site or to a substance produced by or associated with the target site via a primary binding site. The targeting species also has a separate functional group that is capable of forming a bond with the second oligopeptide. In one embodiment, such a bond may be an amide bond formed by the reaction of a carboxylic acid group in the targeting species and the terminal amino group in the second oligopeptide (or the reaction of an amino group in the targeting species and the terminal carboxylic acid group in the second oligopeptide). In a preferred embodiment, the second oligopeptide is the complementary PNA to the first PNA in the active agent-labeled species.

Proteins are known that preferentially bind marker substances that are produced by or associated with lesions. For example, antibodies can be used against cancer-associated substances, as well as against any pathological lesion that shows an increased or unique antigenic marker, such as against substances associated with cardiovascular lesions, for example, vascular clots including thrombi and emboli, myocardial infarctions and other organ infarcts, and atherosclerotic plaques; inflammatory lesions; and infectious and parasitic agents. Examples of appropriate applications are provided in the above-referenced and incorporated Goldenberg patents and applications.

Cancer states include carcinomas, melanomas, sarcomas, neuroblastomas, leukemias, lymphomas, gliomas, myelomas, and neural tumors.

Infectious diseases include those caused by invading microbes or parasites. As used herein, “microbe” denotes virus, bacteria, rickettsia, mycoplasma, protozoa, fungi and like microorganisms, “parasite” denotes infectious, generally microscopic or very small multicellular invertebrates, or ova or juvenile forms thereof, which are susceptible to antibody-induced clearance or lytic or phagocytic destruction, e.g., malarial parasites, spirochetes and the like, including helminths, while “infectious agent” or “pathogen” denotes both microbes and parasites.

The protein substances useful as targeting species in the present invention include protein, peptide, polypeptide, glycoprotein, lipoprotein, or the like; e.g. hormones, lymphokines, growth factors, albumin, cytokines, enzymes, immune modulators, receptor proteins, antibodies and antibody fragments.

The protein substances of particular interest in the present invention are antibodies and antibody fragments. The terms “antibodies” and “antibody fragments” mean generally immunoglobulins or fragments thereof that specifically bind to antigens to form immune complexes.

The antibody may be a whole immunoglobulin of any class; e.g., IgG, IgM, IgA, IgD, IgE, chimeric or hybrid antibodies with dual or multiple antigen or epitope specificities. It can be a polyclonal antibody, preferably an affinity-purified antibody from a human. It can be an antibody from an appropriate animal; e.g., a primate, goat, rabbit, mouse, or the like. If the target site-binding region is obtained from a non-human species, it is preferred that the target species is humanized to reduce immunogenicity of the non-human antibodies, for use in human diagnostic or therapeutic applications. Such a humanized antibody or fragment thereof is also termed “chimeric.” For example, a chimeric antibody comprises non-human (such as murine) variable regions and human constant regions. A chimeric antibody fragment can comprise a variable binding sequence or complementarity-determining regions (“CDR”) derived from a non-human antibody within a human variable region framework domain. Monoclonal antibodies are also suitable for use in the present invention, and are preferred because of their high specificities. They are readily prepared by what are now considered conventional procedures of immunization of mammals with an immunogenic antigen preparation, fusion of immune lymph or spleen cells with an immortal myeloma cell line, and isolation of specific hybridoma clones. More unconventional methods of preparing monoclonal antibodies are not excluded, such as interspecies fusions and genetic engineering manipulations of hypervariable regions, since it is primarily the antigen specificity of the antibodies that affects their utility in the present invention. It will be appreciated that newer techniques for production of monoclonal antibodies (“MAb”) can also be used; e.g., human MAbs, interspecies MAbs, chimeric (e.g., human/mouse) MAbs, genetically engineered antibodies, and the like.

Antibody fragments useful in the present invention include F(ab′)₂, F(ab)₂, Fab′, Fab, Fv, and the like including hybrid fragments. Preferred fragments are Fab′, F(ab′)₂, Fab, and F(ab)₂. Also useful are any subfragments retaining the hypervariable, antigen-binding region of an immunoglobulin and having a size similar to or smaller than a Fab′ fragment. An antibody fragment can include genetically engineered and/or recombinant proteins, whether single-chain or multiple-chain, which incorporate an antigen-binding site and otherwise function in vivo as targeting species in substantially the same way as natural immunoglobulin fragments. Such single-chain binding molecules are disclosed in U.S. Pat. No. 4,946,778. Fab′ antibody fragments may be conveniently made by reductive cleavage of F(ab′)₂ fragments, which themselves may be made by pepsin digestion of intact immunoglobulin. Fab antibody fragments may be made by papain digestion of intact immunoglobulin, under reducing conditions, or by cleavage of F(ab)₂ fragments which result from careful papain digestion of whole immunoglobulin. The fragments may also be produced by genetic engineering.

It should be noted that mixtures of antibodies and immunoglobulin classes can be used, as can hybrid antibodies. Multispecific, including bispecific and hybrid, antibodies and antibody fragments are sometimes desirable in the present invention for detecting and treating lesions and comprise at least two different substantially monospecific antibodies or antibody fragments, wherein at least two of said antibodies or antibody fragments specifically bind to at least two different antigens produced or associated with the targeted lesion or at least two different epitopes or molecules of a marker substance produced or associated with the targeted lesion. Multispecific antibodies and antibody fragments with dual specificities can be prepared analogously to the anti-tumor marker hybrids disclosed in U.S. Pat. No. 4,361,544. Other techniques for preparing hybrid antibodies are disclosed in; e.g., U.S. Pat. Nos. 4,474,893 and 4,479,895, and in Milstein et al., Immunology Today, Vol. 5, 299 (1984).

Preferred are proteins having a specific immunoreactivity to a biomarker substance of at least 60% and a cross-reactivity to other antigens or non-targeted substances of less than 35%.

As disclosed above, antibodies against tumor antigens and against pathogens are known. For example, antibodies and antibody fragments which specifically bind biomarkers produced by or associated with tumors or infectious lesions, including viral, bacterial, fungal and parasitic infections, and antigens and products associated with such microorganisms have been disclosed, inter alia, in Hansen et al. (U.S. Pat. No. 3,927,193) and Goldenberg (U.S. Pat. Nos. 4,331,647, 4,348,376, 4,361,544, 4,468,457, 4,444,744, 4,818,709 and 4,624,846). In particular, antibodies against an antigen, e.g., a gastrointestinal, lung, breast, prostate, ovarian, testicular, brain or lymphatic tumor, a sarcoma, or a melanoma, are advantageously used.

A wide variety of monoclonal antibodies against infectious disease agents have been developed, and are summarized in a review by Polin, in Eur. J. Clin. Microbiol., 3(5):387-398, 1984, showing ready availability. These include MAbs against pathogens and their antigens. Exemplary infectious disease agents are disclosed in U.S. Pat. No. 5,482,698, which is incorporated herein by reference.

Additional examples of MAbs generated against infectious organisms that have been described in the literature are noted below.

MAbs against the gp 120 glycoprotein antigen of human immunodeficiency virus 1 (HIV-1) are known, and certain of such antibodies can have an immunoprotective role in humans. See, e.g., Rossi et al., Proc. Natl. Acad. Sci. USA, Vol. 86, pp. 8055-58 (1990). Other MAbs against viral antigens and viral-induced antigens are also known. MAbs against malaria parasites can be directed against the sporozoite, merozoite, schizont and gametocyte stages.

Suitable MAbs have been developed against most of the microorganisms (bacteria, viruses, protozoa, other parasites) responsible for the majority of infections in humans, and many have been used previously for in vitro diagnostic purposes. These antibodies, and newer MAbs that can be generated by conventional methods, are appropriate for use in the present invention.

Proteins useful for detecting and/or treating cardiovascular lesions include fibrin-specific proteins; for example, fibrinogen, soluble fibrin, antifibrin antibodies and fragments, fragment E₁ (a 60 kDa fragment of human fibrin made by controlled plasmin digestion of crosslinked fibrin), plasmin (an enzyme in the blood responsible for the dissolution of fresh thrombi), plasminogen activators (e.g., urokinase, streptokinase and tissue plasminogen activator), heparin, and fibronectin (an adhesive plasma glycoprotein of 450 kDa) and platelet-directed proteins; for example, platelets, antiplatelet antibodies, and antibody fragments, anti-activated platelet antibodies, and anti-activated platelet factors, which have been reviewed by Koblik et al., Semin. Nucl. Med., Vol. 19, 221-237 (1989), which is incorporated herein by reference.

According to one embodiment of the present invention, the targeting species is an MAb or a fragment thereof that recognizes and binds to a heptapeptide of the amino terminus of the β-chain of fibrin monomer. Fibrin monomers are produced when thrombin cleaves two pairs of small peptides from fibrinogen. Fibrin monomers spontaneously aggregate into an insoluble gel, which is further stabilized to produce blood clots. The second oligopeptide of the pretargeting conjugate of the present invention is attached to the MAb or fragment thereof containing the binding site for this heptapeptide can provide an effective agent for the detection, localization, or therapy of deep vein and coronary artery thrombi. Such heptapeptide and MAb are disclosed in K. Y. Hui et al., “Monoclonal Antibodies to a Synthetic Fibrin-Like Peptide Bind to Human Fibrin but Not Fibrinogen,” Science, Vol. 222, 1129 (1983). In a preferred embodiment, the second oligopeptide is a PNA complementary to the first PNA of the active agent-labeled species.

According to another embodiment of the present invention, the targeting species is a chimeric antibody derived from an antibody designated as NR-LU-10. This chimeric antibody has been designated as NR-LU-13 and disclosed in U.S. Pat. No. 6,358,710, which is incorporated herein in its entirety by reference. NR-LU-13 contains the murine Fv region of NR-LU-10 and therefore comprises the same binding specificity as NR-LU-10. It also comprises human constant regions. Thus, this chimeric antibody binds the NR-LU-10 antigen and is less immunogenic because it is made more human-like. NR-LU-10 is a nominal 150 kilodalton (or kDa) murine IgG2b pan carcinoma monoclonal antibody that recognizes an approximately 40 kDa glycoprotein antigen expressed on most carcinomas, such as small cell lung, non-small cell lung, colon, breast, renal, ovarian, pancreatic, and other carcinoma tissues. The NR-LU-10 antigen has been further described by Varki et al., “Antigens Associated With a Human Lung Adenocarcinoma Defined by Monoclonal Antibodies,” Cancer Research, Vol. 44, 681-87 (1984), and Okabe et al., ““Monoclonal Antibodies to Surface Antigens of Small Cell carcinoma of the Lungs,” Cancer Research Vol. 44, 5273-78 (1984). Methods for preparing antibodies that binds to epitopes of the NR-LU-10 antigen are known and are disclosed in U.S. Pat. No. 5,084,396, which is incorporated herein in its entirety by reference. One suitable method for producing monoclonal antibodies is the standard hybridoma production and screening process, which is well known in the art. In a preferred embodiment, the targeting species is a humanized antibody or humanized antibody fragment that binds specifically to the antigen bound by antibody NR-LU-13. A humanization method comprises grafting only non-human CDRs onto human framework and constant regions (see; e.g., Jones et al., Nature, Volume 321, 522-35 (1986)). Another humanization method comprises transplanting the entire non-human variable domains, but cloaking (or veneering) these domains by replacement of exposed residues reduce immunogenicity (see; e.g., Padlan, Molec. Immun., Vol. 28, 489-98 (1991)). Exemplary humanized light and heavy sequences derived from the light and heavy sequences of the NR-LU-13 antibody are disclosed in U.S. Pat. No. 6,358,710, and are denoted therein as NRX451. The phrase “binds specifically” with respect to antibody or antibody fragment means such antibody or antibody fragment has a binding affinity of at least about 10⁴ M⁻¹. Preferably, the binding affinity is at least about 10⁶ M⁻¹, and more preferably, at least about 10⁸ M⁻¹.

According to still another embodiment, the targeting species is a humanized anti-p185^{HER2} antibody that specifically recognizes the p185 HER2 protein expressed on breast cancer cells. A humanized anti-p185^{HER2} antibody known as Herceptin is widely available. An anti-HER2 murine MAb known as ID5 is available from Applied BioTechnology/Oncogene Science (Cambridge, Mass.), which can be humanized according to conventional methods. See, e.g., X. F. Lee et al., “Differential Signaling by an Anti-p185^{HER2} Antibody and Hergulin,” Cancer Research, Vol. 60, 3522-31 (2000).

In other embodiments of the present invention, the targeting species is an antibody or a fragment thereof, preferably a humanized antibody or fragment thereof, that is raised against one of anti-carcinogembryonic antigen (“CEA”), anti-colon-specific antigen-p (“CSAp”), and other well known tumor-associated antigens, such as CD19, CD20, CD21, CD22, CD23, CD30, CD74, CD80, HLA-DR, I, MUC 1, MUC 2, MUC 3, MUC 4, EGFR, HER2/neu, PAM4, Bre3, TAG-72 (C72.3, CC49), EGP-1 (e.g., RS7), EGP-2 (e.g., 17-1A and other Ep-CAM targets), Le(y (e.g., B3), A3, KS-1, S100, IL-2, T101, necrosis antigens, folate receptors, angiogenesis markers (e.g., VEGFR), tenascin, PSMA, PSA, tumor-associated cytokines, MAGE and/or fragments thereof. Tissue-specific antibodies (e.g., against bone marrow cells, such as CD34, CD74, etc., parathyroglobulin antibodies, etc.) as well as antibodies against non-malignant diseased biomarkers, such as macrophage antigens of atherosclerotic plaques (e.g., CD74 antibodies), and also specific pathogen antibodies (e.g., against bacteria, viruses, and parasites) are well known in the art.

It should be understood that the foregoing disclosure of various antigens or biomarkers that can be used to raise specific antibodies against them (and from which antibodies fragments may be prepared) serves only as examples, and is not to be construed in any way as a limitation of the present invention.

Conjugating or Attaching Targeting Species to an Oligopeptide

Targeting species are conjugated or otherwise attached to the second oligopeptide to produce the pretargeting conjugate. For example, the conjugation or attachment can be effected via an amide bond or a disulfide bond. Targeting species that are polypeptides, such as an antibody or an antibody fragment, are conjugated to the second oligopeptide via amide bonds between free amino groups of lysine residues present in the antibody or the antibody fragment and the terminal carboxylic group of the second oligopeptide. Alternatively, amide bonds can be formed between free carboxylic groups of glutamic acid or aspartic acid residues present in the antibody or the antibody fragment and the terminal amino group of the second oligopeptide. The conjugation process can be carried out by contacting the antibody or antibody fragment and the second oligopeptide at pH in the range from about 7 to about 9.5 in a buffer, at or near room temperature. At the end of the reaction period, the conjugate is separated from the unreacted low molecular-weight materials by, for example, size exclusion chromatography and/or dialysis.

The second oligopeptide can also be conjugated to the antibody or antibody fragment via disulfide bonds formed with cysteine residues present in the antibody or antibody fragment. In this case, a cysteine residue is first attached to an end of the second oligopeptide to provide a mercapto group thereto. The second oligopeptide, as modified, is then reacted with the antibody or antibody fragment to produce the pretargeting conjugate as above.

Alternatively, carbohydrate moieties present in the antibody or antibody fragment may be oxidized mildly, such as with sodium metaperiodate at or near room temperature. Unreacted sodium metaperiodate may be decomposed with ethylene glycol. The oxidized antibody or antibody fragment is then separated from low molecular-weight materials, for example, by size exclusion chromatography, and subsequently reacted with the second oligopeptide to produce the pretargeting conjugate.

Synthesis of PNA Monomers and Oligopeptides

In a preferred embodiment of the present invention, the first and the second oligopeptides are PNAs that are complementary to one another. PNA monomers and oligopeptides were prepared according to the method disclosed below.

Overall scheme for the synthesis of peptide monomer backbone, ethyl-N(2-Boc-aminoacetyl) glycinate, is shown below:

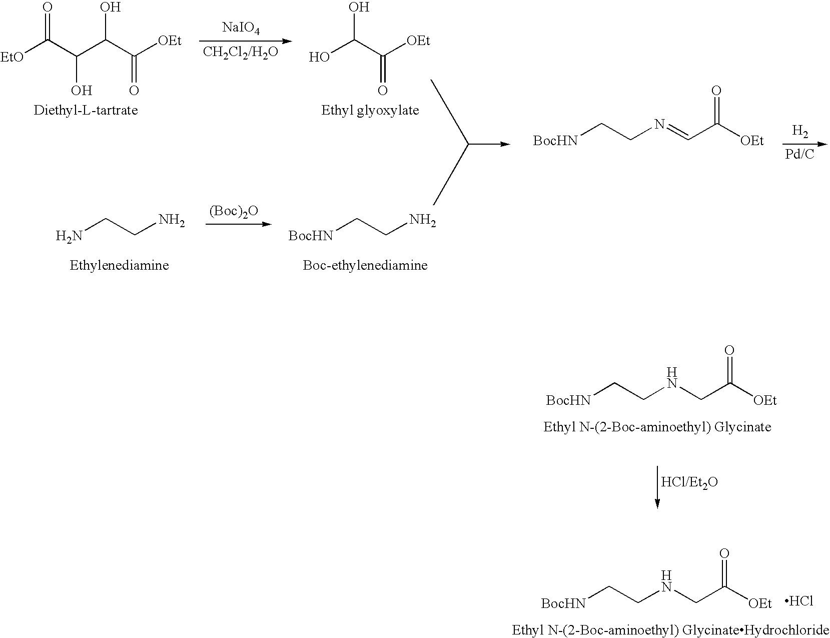

Boc-ethylenediamine: A 250 ml three-neck round bottom flask was equipped with mechanical stirrer and charged with 7.0 ml (6.25 g, 104 mmol, 3.5M) of ethylenediamine, 30 ml of THF and cooled to 0° C. To this was added 7.57 g (34.7 mmol, 1.1 M) of di-tert-butyl dicarbonate in 30 ml of THF dropwise over 30 min with rapid stirring. After addition was complete, the solution was stirred at 0° C. for 30 min and at ambient temperature overnight. Volatiles were removed under rotary evaporation and the residue was placed between ethyl acetate and brine. The organic layer was washed with brine, dried with Na₂SO₄ to yield 13.0 g (81 mmol, 78%) of a clear oil.

Ethyl glyoxylate hydrate: A 500 ml three-neck round bottom flask was equipped with reflux condenser and charged with 20.9 g (101 mmol) of diethyl-L-tartrate and 200 ml of CH₂Cl₂. Solid sodium periodate, 43.4 g (203 mmol), was added in small portions with rapid stirring, followed by 40 ml of H₂O. The mixture was refluxed with rapid stirring for 2 hr. during which a thick precipitate was formed. This was cooled to 0° C. and 80 g of MgSO₄ was added in small portions over 20 min. The mixture was continued to stir for an additional 15 min. This was filtered and washed with CH₂Cl₂. All volatiles were removed under rotary evaporation to yield 21.9 g (90%) of a clear oil.

Ethyl N-(2-Boc-aminoethyl) glycinate: Boc-ethylenediamine, 9.47 g (160 mmol) in 15 ml CH₂Cl₂, was added dropwise over 10 min to a stirred 0° C. solution of to 7.81 g (65 mmol) of ethyl glyoxylate hydrate and 8 g of (oven dried) 3 Å molecular sieves. After stirring for 1 hr. at 0° C. the mixture was filtered through a tightly packed plug of Celite placed on a fritted filter. The clear solution was placed in a glass liner and 3.2 g of 10% Pd/C (3.0 mmol Pd) was added. This was placed in a high pressure reactor and the mixture was hydrogenated at 50 psig and 30° C. for 4 hr. The resulting mixture was filtered through a tightly packed plug of Celite placed on a fritted filter and washed with isopropanol. All volatiles were removed by rotary evaporation to yield 9.62 g (39 mmol, 66%) of a clear oil.

Ethyl N-(2-Boc-aminoethyl) glycinate hydrochloride. To a solution of ethyl N-(2-Boc-aminoethyl) glycinate, 9.62 g (39 mmol) in 165 ml of anhydrous ether at 0° C. was added dropwise 42 ml of ethereal HCl (1.0M, 42 mmol) over 10 min. The mixture was mechanically stirred at 0° C. for 1 hour. This was filtered, washed with ether, volatiles removed by rotary evaporation and dried under high vacuum to yield 8.48 g (30 mmol, 77%) of a fine white powder.

Overall scheme for the synthesis of PNA monomers is shown below:

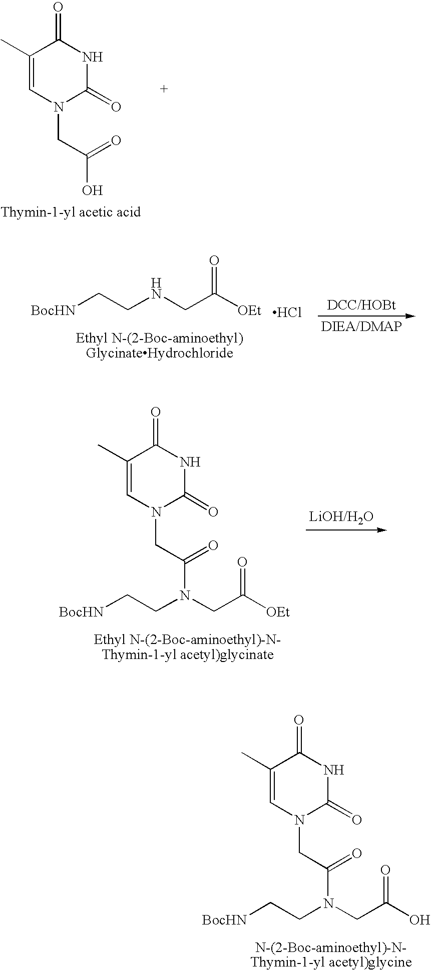

In the foregoing reaction, thymine is used to illustrate the synthesis of a PNA monomer containing this base. However, it should be understood that another starting substituted acetic acid may be used that carries the desired heterocyclic base (adenine, guanine, cytosine, or uracil). See; e.g., Dueholm et al., “Synthesis of Peptide Nucleic Acid Monomers Containing the Four Natural Nucleobases: Thymine, Cytosine, Adenine, and Guanine and Their Oligomization,” J. Org. Chem., Vol. 59, 5767-73 (1994).

General Synthesis of PNA Monomers: To a solution of 2.0 g (10.9 mmol) of thymin-1-yl acetic acid and 1.62 g (12 mmol) of 1-hydroxybenzotriazole in 30 ml anhydrous DMF at 0° C. was added 2.48 g (12 mmol) of N,N-dicyclohexylcarbodiimide. This was warmed to ambient temperature and stirred for 2 hours. This was cooled to 0° C. and a solution of 3.39 g (12 mmol) of ethyl N-(2-Boc-aminoethyl)glycinate hydrochloride, 6.3 ml (4.67 g, 36 mmol) of diisopropylethylamine and 134 mg (1.1 mmol) of dimethylaminopyridine in 200 ml of anhydrous DMF was added. The mixture was warmed to ambient temperature and mechanically stirred overnight. This was filtered, the precipitate washed with 2×15 ml CH₂Cl₂. To the combined filtrates was added an additional 120 ml of CH₂Cl₂ and the solution was washed with 3×100 ml of dilute aqueous NaHCO₃, 2×100 ml dilute aqueous KHSO₄ and 100 ml of brine. The precipitate in the organic phase was filtered, the filtrate dried over Na₂SO₄. All volatiles were removed under rotary evaporation and the resulting reside was dried under high vacuum. This was redissolved in 25 ml of CH₂Cl₂ and the desired ethyl N-(2-Boc-aminoethyl)-N-(thymin-1-yl acetyl)glycinate was precipitated with addition of 65 ml of hexanes. This was redissolved in another portion of CH₂Cl₂ and again precipitated with hexanes and dried under high vacuum to yield 3.31 g (8.61 mmol, 79%) of a white powder.

The N-(2-Boc-aminoethyl)-N-(thymin-1-yl acetyl)glycinate, 3.31 g (8.61 mmol,) was suspended in 40 ml THF and to it 26 ml of a 1.0M aqueous LiOH (26 mmol) was added and the mixtures stirred at ambient temperature for 1 hour. This was filtered and to the filtrate was added an additional 10 ml H₂O. This was washed with 60 ml CH₂Cl₂. More water (6 ml) was added and again washed with 30 ml CH₂Cl₂. The aqueous layer was cooled to 0° C. and 1M HCl was added dropwise until the pH reached 2. This was extracted with 9×50 ml ethyl acetate, volatiles removed by rotary evaporation and dried under high vacuum to yield 2.2 g (6.2 mmol, 72%) of a white powder. Over yield of the combined steps was 57%).

PNA oligopeptides can be prepared from the monomers prepared by the method disclosed above by following standard solid-phase synthesis protocols for peptides using, for example, a (methyl-benzhydryl)amine polystyrene resin as the solid support. Such a method is disclosed, for example, in Merrifield, “Solid-Phase Synthesis. I. The Synthesis of a Tetrapeptide,” J. Am. Chem. Soc., Vol. 85, 2149-54 (1963); Merrifield, “Solid-Phase Synthesis,” Science, Vol. 232, 341-47 (1986); and Christensen et al., “Solid-Phase Synthesis of Peptide Nucleic Acids,” J. Peptide Sci., Vol. 3, 175-83 (1995).

Moreover, a PNA oligopeptide, as prepared above, can be further modified to provide a coupling moiety useful for subsequent conjugation to a targeting species, such as an antibody or antibody fragment. Such further modification can comprise attaching a lysine or cysteine residue to a terminus of the PNA oligopeptide to provide a free amino or a mercapto group for such subsequent conjugation.

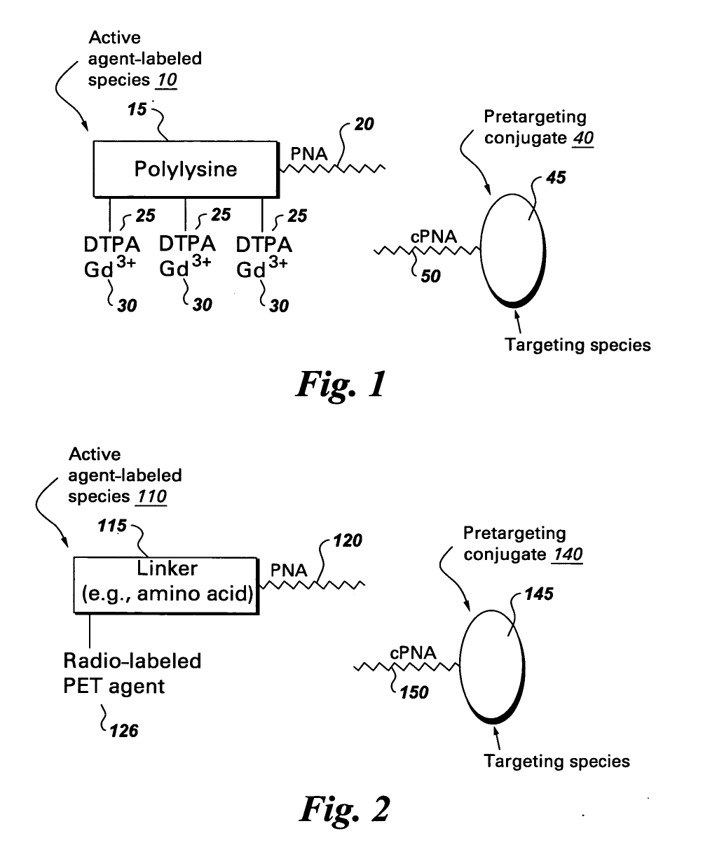

FIG. 1 shows schematically a preferred embodiment of the present invention that is suitable for MRI diagnostic imaging applications. Active agent-labeled species 10 comprises polylysine 15, which is conjugated to a plurality of chelating moieties 25, such as DTPA. Chelating moieties 25 form coordination complexes with paramagnetic ions 30, such as Gd³⁺ ions. Preferably, polylysine 15 comprises from about 100 to about 600 lysine residues, and has a degree of conjugation with chelating moieties of at least 90 percent. Such a degree of conjugation imparts an extended conformation to polylysine 15, which conformation allows active agent-labeled species 10 to penetrate many small spaces within a diseased tissue. Polylysine 15 is attached at one end to first oligopeptide 20, such as a PNA sequence. Pretargeting conjugate 40, together with active agent-labeled species 10, form the pair of compounds of the present invention designed to be used together to elucidate a diseased cell or tissue. Pretargeting conjugate 40 comprises targeting species 45, such as an antibody or a fragment thereof that is capable to bind to a target site (e.g., diseased cell or tissue), or a substance expressed by the target site, and a second oligopeptide 50 (e.g., complementary PNA) that is complementary to first oligopeptide 20.

FIG. 2 shows schematically a preferred embodiment of the present invention that is suitable for PET diagnostic imaging applications. Active agent-labeled species 110 comprises linker 115, which is conjugated to one or more radioactive-labeled moieties 126, such as those emitting positrons. Linker 115 can comprise from about 1 to about 20 amino acid residues. In one embodiment, linker 115 may be entirely eliminated. In such a case, radioactive-labeled moiety 126 is attached directly to first oligopeptide 120, such as a PNA sequence. When linker 115 has a finite length, it is attached to one end of first oligopeptide 120. Pretargeting conjugate 140, together with active agent-labeled species 110, form the pair of compounds of the present invention designed to be used together to elucidate a diseased cell or tissue. Pretargeting conjugate 140 comprises targeting species 145, such as an antibody or a fragment thereof that is capable of binding to a target site (e.g., diseased cell or tissue), or a substance expressed by the target site, and a second oligopeptide 150 (e.g., complementary PNA) that is complementary to first oligopeptide 120.

The compounds of the present invention, for example, the active agent-labeled species, the pretargeting conjugate, or both, can be incorporated into pharmaceutical compositions suitable for administration into a subject, which pharmaceutical compositions comprise a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible with the subject. Preferably, the carrier is suitable for intravenous, intramuscular, subcutaneous, or parenteral administration (e.g., by injection). Depending on the route of administration, the active agent-labeled species, the pretargeting conjugate, or both, may be coated in a material to protect the compound or compounds from the action of acids and other natural conditions that may inactivate the compound or compounds.

In yet another embodiment of the present invention, the pharmaceutical composition comprising the active agent-labeled species, the pretargeting conjugate, or both, and a pharmaceutically acceptable carrier can be administered by combination therapy, i.e., combined with other agents. For example, the combination therapy can include a composition of the present invention with at least a therapeutic agent or drug, such as an anti-cancer or an antibiotic. Exemplary anti-cancer agents include cis-platin, adriamycin, and taxol. Exemplary antibiotics include isoniazid, rifamycin, and tetracycline.

Methods for Diagnosing or Treating Diseases Using Pretargeting Strategy

The present invention provides a method for detecting, diagnosing, and/or treating a disease condition by preferentially delivering an active agent (diagnostic, therapeutic, or both) to the site of the disease. A pair of compounds or pharmaceuticals comprising a pretargeting conjugate and an active agent-labeled species is administered into a patient in a method of the present invention for diagnosing or treating a disease. A patient can be human or non-human. In general, the method comprises: (a) administering a pretargeting conjugate into the patient or subject, wherein the pretargeting conjugate comprises: (1) a targeting species having a targeting moiety that binds to a target or a marker substance produced by or associated with the target; and (2) a second oligopeptide that is complementary to a first oligopeptide; (b) allowing the pretargeting conjugate to localize at the target; and (c) administering an active agent-labeled species into the patient or subject, wherein the active agent-labeled species comprises the active agent conjugated to the first oligopeptide.

In one embodiment, the targeting species is an antibody or a fragment thereof that binds to an antigen present at the target, which can be a diseased cell or tissue, or a marker substance produced by the diseased cell or tissue. The active agent is a moiety that generate a unique signal that is recognizable by diagnostic medical imaging techniques, such as MRI, PET, SPECT, X-ray imaging, CT, ultrasound imaging, or optical imaging.

In another embodiment, the active agent is a radioisotope, drug, toxin, fluorescent dye activated by nonionizing radiation, hormone, hormone antagonist, receptor antagonist, enzyme or proenzyme activated by another agent, autocrine, or cytokine.

In one aspect, the active agent-labeled species comprises a poly(amino acid) (e.g., as polylysine), wherein at least 90 percent of the lysine residues are conjugated to chelating moieties, (e.g., DTPA), which form coordination complex with paramagnetic ions, (e.g., Gd³⁺) for MRI application. The active agent-labeled species may be administered into the patient at a dose from about 0.01 to about 0.05 moles Gd/kg of body weight of the patient. An MRI system that can be used for practicing a method of the present invention is disclosed in U.S. Pat. No. 6,235,264; which is incorporated herein by reference in its entirety. The pair of pharmaceuticals of the present invention is formulated with a physiologically acceptable carrier, such as an intravenous fluid, for intravenously administering into the patient. These pharmaceuticals may also be administered orally under appropriate circumstances.

In another aspect, a method for diagnosing a disease condition comprises: (a) obtaining at least a base-line image of and acquiring a base-line signal from a portion of a subject, which portion is suspected to carry the disease; (b) administering a pretargeting conjugate into the patient or subject, wherein the pretargeting conjugate comprises: (1) a targeting species having a targeting moiety that binds to a target or a marker substance produced by or associated with the target; and (2) a second oligopeptide that is complementary to a first oligopeptide; (c) allowing the pretargeting conjugate to localize at the target; (d) administering an active agent-labeled species into the patient or subject, wherein the active agent-labeled species comprises the active agent conjugated to the first oligopeptide; and (e) obtaining an additional image of and acquiring an additional signal from the same portion of the subject. Any difference between such base-line image and such additional image indicates the presence or condition of the disease (e.g., the state or the spread).

In another aspect, the present invention provides a method for assessing an effectiveness of a prescribed regimen for treating a disease that is characterized by an overproduction of a disease-specific substance or biomarker. The method comprises: (a) obtaining at least a base-line image of and acquiring a base-line signal from a portion of a subject, which portion is suspected to carry the disease; (b) administering a pretargeting conjugate into the patient or subject, wherein the pretargeting conjugate comprises: (1) a targeting species having a targeting moiety that binds to a target or a marker substance produced by or associated with the target; and (2) a second oligopeptide that is complementary to a first oligopeptide; (c) allowing the pretargeting conjugate to localize at the target; (d) administering an active agent-labeled species into the patient or subject, wherein the active agent-labeled species comprises the active agent conjugated to the first oligopeptide; (e) obtaining pre-treatment images of and acquiring pre-treatment signals coming from the portion of the subject, which portion is suspected to carry the disease, after steps (b), (c), and (d); (f) treating a condition of the disease in the subject with the prescribed regimen; (g) repeating steps (b), (c), and (d); and (h) obtaining post-treatment images of and acquiring post-treatment signals coming from the same portion of the subject as in step (e); and (i) comparing post-treatment images and post-treatment signals to pre-treatment images and pre-treatment signals to assess the effectiveness of the prescribed regimen. A decrease in image contrast or signals during the course of the prescribed regimen indicates that the treatment has provided benefit. The method further comprises repeating steps (h) and (i) at predetermined time intervals during the course of treatment of the disease.

In various aspects of the method of the present invention, any one of the pretargeting conjugates and active agent-labeled species that are specifically described above can be chosen to suit the particular circumstances and disease.

During the course of the treatment of the disease, a decreased signal obtained from the imaging technique (compared to a base-line signal obtained before the treatment) of, for example, 10 percent or more can signify that the treatment has conferred some benefit. In another embodiment, a decreased signal obtained from the imaging technique (compared to a base-line signal obtained before the treatment) of, for example, 20 percent or more can signify that the treatment has conferred some benefit. The prescribed regimen for treating the disease can be, for example, treatment with drugs, radiation, or surgery.

While various embodiments are described herein, it will be appreciated from the specification that various combinations of elements, variations, equivalents, or improvements therein may be made by those skilled in the art, and are still within the scope of the invention as defined in the appended claims.

1. A set of compounds comprising a first compound and a second compound, wherein the first compound comprises a first oligopeptide that is conjugated to a linker having a first moiety for coupling with an active agent selected from the group consisting of diagnostic active agents and therapeutic active agents, the second compound comprises a second oligopeptide that is conjugated to a targeting species having a targeting moiety capable of binding to an in-vivo target, and the second oligopeptide comprises a sequence complementary to a sequence of the first oligopeptide.

2. The set of compounds according to claim 1, wherein the first oligopeptide is a first peptide nucleic acid (“PNA”) sequence having a formula of 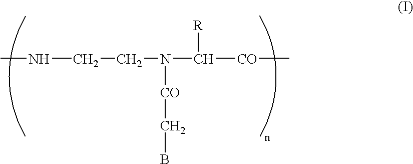
wherein B is a heterocyclic base selected from the group consisting of adenine, guanine, cytosine, thymine, and uracil; R is selected from the group consisting of side groups covalently bonded to α-carbons of twenty known α-amino acids; and n is an integer in a range from 4 to 20, inclusive; and wherein the second oligopeptide is a second PNA sequence that is complementary to the first PNA sequence.

3. The set of compounds according to claim 2, wherein n is an integer in a range from 6 to 14, inclusive.

4. The set of compounds according to claim 2, wherein the linker is a poly(amino acid), the first moiety is a chelating moiety that is conjugated to the poly(amino acid), and the active agent is a diagnostic agent capable of generating a signal that is detectable by a technique selected from the group consisting of magnetic resonance imaging (“MRI”), positron emission tomography (“PET”), single photon emission computed tomography (“SPECT”), compute tomography, X-ray imaging, ultrasound, and optical imaging.

5. The set of compounds according to claim 4, wherein the poly(amino acid) is polylysine.

6. The set of compounds according to claim 4, wherein the chelating moiety is selected from the group consisting of diethylenetriamine-pentaacetic acid (“DTPA”), 1,4,7,10-tetraazacyclododecane-N,N′,N″,N′″-tetraacetic acid (“DOTA”), p-isothiocyanatobenzyl-1,4,7,10-tetraazacyclododecane-1,4,7,10-tetraacetic acid (“p-SCN-Bz-DOTA”), 1,4,7,10-tetraazacyclododecane-N,N′,N″-triacetic acid (“DO3A”), 1,4,7,10-tetraazacyclododecane-1,4,7,10-tetrakis(2-propionic acid) (“DOTMA”), 3,6,9-triaza-12-oxa-3,6,9-tricarboxymethylene-10-carboxy-13-phenyl-tridecanoic acid (“B-19036”), 1,4,7-triazacyclononane-N,N′,N″-triacetic acid (“NOTA”), 1,4,8,11-tetraazacyclotetradecane-N,N′,N″,N′″-tetraacetic acid (“TETA”), triethylene tetreamine hexaacetic acid (“TTHA”), trans-1,2-diaminohexane tetraacetic acid (“CYDTA”), 1,4,7,10-tetraazacyclododecane-1-(2-hydroxypropyl)4,7,10-triacetic acid (“HP-DO3A”), trans-cyclohexane-diamine tetraacetic acid (“CDTA”), trans(1,2)-cyclohexane diethylene triamine pentaacetic acid (“CDTPA”), 1-oxa-4,7,10-triazacyclododecane-N,N′,N″-triacetic acid (“OTTA”), 1,4,7,10-tetraazacyclododecane-1,4,7,10-tetrakis{3-(4-carboxyl)-butanoic acid}, 1,4,7,10-tetraazacyclododecane-1,4,7,10-tetrakis(acetic acid-methyl amide), 1,4,7,10-tetraazacyclododecane-1,4,7,10-tetrakis(methylene phosphonic acid), and derivatives thereof; and the chelating moiety forms a coordination complex with a paramagnetic species.

7. The set of compounds according to claim 5, wherein the poly(amino acid) is polylysine having from about 100 to about 600 lysine residues, and wherein at least ninety percent of the lysine residues are conjugated to the chelating moiety.

8. The set of compounds according to claim 4, wherein the active agent is paramagnetic Gd³⁺.

9. The set of compounds according to claim 2, wherein the active agent is paramagnetic iron oxide that is coupled to the linker.

10. The set of compounds according to claim 2, wherein the linker is coupled to an active agent comprising an active-agent moiety that generates a signal detectable by a technique selected from the group consisting of PET and SPECT.

11. The set of compounds according to claim 10, wherein the active-agent moiety comprises an isotope selected from the group consisting of F-1B, I-123, I-124, I-125, Cu-64, and Cu-67.

12. The set of compounds according to claim 2, wherein the active agent is a therapeutic agent selected from the group consisting of isotopes, drugs, toxins, fluorescent dyes activated by nonionizing radiation, hormones, hormone antagonists, receptor antagonists, enzymes or proenzymes activated by another agent, autocrine, and cytokine.

13. The set of compounds according to claim 12, wherein the therapeutic agent is selected from the group consisting of taxol, nitrogen mustards, cyclophosphamide, melphalan, uracil mustard, chlorambucil, ethylenimine derivatives, alkyl sulfonates, nitrosoureas, triazenes, folio acid analogs, pyrimidine analogs, purine analogs, vinca alkaloids, antibiotics, enzymes, platinum coordination complexes, substituted urea, methyl hydrazine derivatives, adrenocortical suppressants, hormones, and antagonists.

14. The set of compounds according to claim 1, wherein the targeting species is selected from the group consisting of proteins, peptides, polypeptides, glycoproteins, lipoproteins, phospholipids, oligonucleotides, steroids, hormones, lymphokines, growth factors, albumin, cytokines, enzymes, immune modulators, receptor proteins, antisense oligonucleotides, antibodies, and antibody fragments, which targeting moiety is capable of binding biomarkers that are produced by or associated with the target.

15. The set of compounds according to claim 14, wherein the targeting species is selected from the group consisting of antibodies and fragments thereof, which targeting species comprises a binding region for a target site or a biomarker produced by or associated with the target.

16. The set of compounds according to claim 15, wherein the targeting species is selected from the group consisting of humanized antibodies and humanized antibody fragments.

17. The set of compounds according to claim 15, wherein the biomarker is associated with a target selected from the group consisting of tumors, cardiovascular lesions, vascular clots, thrombi, emboli, myocardial infarctions, atherosclerotic plaques, inflammatory lesions; and infectious and parasitic agents.

18. A compound comprising a PNA sequence having a formula of 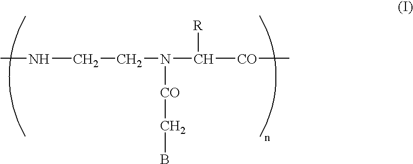
wherein B is a heterocyclic base selected from the group consisting of adenine, guanine, cytosine, thymine, and uracil; R is selected from the group consisting of side groups covalently bonded to α-carbons of twenty known α-amino acids; n is an integer in a range from 4 to 20, inclusive; and the PNA sequence is covalently linked to a poly(amino acid), which is conjugated to a plurality of chelating moieties capable of coupling with an active agent selected from the group consisting of diagnostic agent and therapeutic agent.

19. The compound according to claim 18, wherein the poly(amino acid) is polylysine having from about 100 to about 600 lysine residues, the chelating moieties comprise polycarboxylic acids, at least 90 percent of the lysine residues are conjugated to the chelating moieties, and the chelating moieties form coordination complexes with a paramagnetic material.

20. A compound comprising a PNA sequence having a formula of 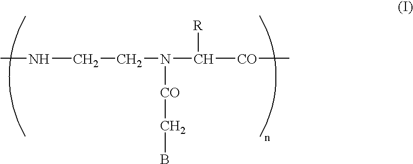
wherein B is a heterocyclic base selected from the group consisting of adenine, guanine, cytosine, thymine, and uracil; R is selected from the group consisting of side groups covalently bonded to α-carbons of twenty known α-amino acids; n is an integer in a range from 4 to 20, inclusive; and the PNA sequence is covalently linked to a linker, which is conjugated to an active-agent moiety capable of generating a signal detectable by a technique selected from the group consisting of PET and SPECT.

21. The compound according to claim 20, wherein the active-agent moiety comprises an isotope selected from the group consisting of F-18, I-123, I-124, I-125, Cu-64, and Cu-67.

22. A method for diagnosing or treating a disease condition, the method comprising:
(a) administering a pretargeting conjugate into a subject, wherein the pretargeting conjugate comprises: (1) a targeting species having a targeting moiety that binds to a target or a marker substance produced by or associated with the target; and (2) a second peptide nucleic acid (“PNA”) sequence that is complementary to a first PNA sequence;
(b) allowing the pretargeting conjugate to localize at the target; and
(c) administering an active agent-labeled species into the subject, wherein the active agent-labeled species comprises the active agent conjugated to the first PNA sequence, and the active agent is capable of performing a function selected from the group consisting of elucidating the disease condition and reducing an adverse effect of the disease condition.

23. The method according to claim 22, wherein the active agent is capable of generating a signal that is detectable by a technique selected from the group consisting of MRI, PET, SPECT, X-ray imaging, CT, ultrasound imaging, and optical imaging.

24. The method according to claim 22, wherein the active agent is a therapeutic agent selected from the group consisting of radioisotopes, drugs, toxins, fluorescent dyes activated by nonionizing radiation, hormones, hormone antagonists, receptor antagonists, enzymes, proenzymes activated by another agent, authorizes, and cytokines.

25. A method for diagnosing a disease condition, the method comprising:
(a) obtaining at least a base-line image of and acquiring a base-line signal from a portion of a subject, which portion is suspected to have the disease condition;
(b) administering a pretargeting conjugate into the subject, wherein the pretargeting conjugate comprises: (1) a targeting species having a targeting moiety that binds to a target or a marker substance produced by or associated with the target; and (2) a second peptide nucleic acid (“PNA”) sequence that is complementary to a first PNA sequence;
(c) allowing the pretargeting conjugate to localize at the target;
(d) administering an active agent-labeled species into the subject, wherein the active agent-labeled species comprises the active agent conjugated to the first PNA sequence, and the active agent is capable of performing a function selected from the group consisting of elucidating the disease condition and reducing an adverse effect of the disease condition;
(e) obtaining an additional image of and acquiring an additional signal from the same portion of the subject; and
(f) comparing the base-line image and base-line signal with the additional image and additional signal to evaluate the disease condition.

26. A method For assessing an effectiveness of a prescribed regimen for treating a disease condition that is characterized by an overproduction of a disease-specific substance or biomarker, the method comprising:
(a) obtaining at least a baseline image of and acquiring a base-line signal from a portion of a subject, which portion is suspected to carry the disease;
(b) administering a pretargeting conjugate into the subject, wherein the pretargeting conjugate comprises: (1) a targeting species having a targeting moiety that binds to a target or a marker substance produced by or associated with the target; and (2) a second PNA sequence that is complementary to a first PNA sequence;
(c) allowing the pretargeting conjugate to localize at the target;
(d) administering an active agent-labeled species into the subject, wherein the active agent-labeled species comprises the active agent conjugated to the first PNA sequence;
(e) obtaining pre-treatment images of and acquiring pre-treatment signals coming from the same portion of the subject;
(f) treating the disease condition in the subject with the prescribed regimen;
(g) repeating steps (b), (c), and (d); and
(h) obtaining post-treatment images of and acquiring post-treatment signals coming from the same portion of the subject as in step (e); and
(i) comparing post-treatment images and post-treatment signals to pretreatment images and pre-treatment signals to assess the effectiveness of the prescribed regimen, wherein a decrease in image contrast or signals during a course of the prescribed regimen indicates that the treatment has provided benefit.

27. The method according to claim 26, further comprising repeating steps (h) and (i) at predetermined time intervals during the course of treatment of the disease.

28. A set of pharmaceutical compositions comprising a first pharmaceutical composition and a second pharmaceutical composition, wherein the first pharmaceutical composition comprises a first pharmaceutically acceptable carrier and a first compound that comprises a first oligopeptide that is conjugated to a linker having a first moiety for coupling with an active agent selected from the group consisting of diagnostic active agents and therapeutic active agents; the second pharmaceutical composition comprises a second pharmaceutically acceptable carrier and a second compound that comprises a second oligopeptide that is conjugated to a targeting species having a targeting moiety capable of binding to an in-vivo target, wherein the second oligopeptide comprises a sequence complementary to a sequence of the first oligopeptide.

29. The set of pharmaceutical compositions of claim 28, wherein the first oligopeptide is a first peptide nucleic acid (“PNA”) sequence having a formula of 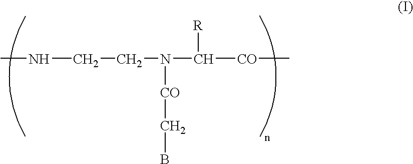
wherein B is a heterocyclic base selected from the group consisting of adenine, guanine, cytosine, thymine, and uracil; R is selected from the group consisting of side groups covalently bonded to α-carbons of twenty known α-amino acids; and n is an integer in a range from 4 to 20, inclusive; and wherein the second oligopeptide is a second PNA sequence that is complementary to the first PNA sequence.

30. The set of pharmaceutical compositions of claim 29, wherein the linker is a poly(amino acid), the first moiety is a chelating moiety that is conjugated to the poly(amino acid), and the active agent is a diagnostic agent capable of generating a signal that is delectable by a technique selected from the group consisting of magnetic resonance imaging (“MRI”), positron emission tomography (“PET”), single photon emission computed tomography (“SPECT”), compute tomography, X-ray imaging, ultrasound, and optical imaging.

31. The set of pharmaceutical compositions of claim 30, wherein the poly(amino acid) is polylysine.

32. The set of pharmaceutical compositions of claim 31, wherein the poly(amino acid) is polylysine having from about 100 to about 600 lysine residues, and wherein at least ninety percent of the lysine residues are conjugated to the chelating moiety.

33. The set of pharmaceutical compositions of claim 30, wherein the active agent is paramagnetic Gd³⁺.

34. The set of pharmaceutical compositions of claim 30, wherein the active agent is paramagnetic iron oxide that is coupled to the linker.

35. The set of pharmaceutical compositions of claim 30, wherein the linker is coupled to an active agent comprising an active-agent moiety that generates a signal detectable by a technique selected from the group consisting of PET and SPECT.

36. The set of pharmaceutical compositions of claim 35, wherein the active-agent moiety comprises an isotope selected from the group consisting of F-18, I-123, I-124, I-125, Cu-64, and Cu-67.

37. The set of pharmaceutical compositions of claim 30, wherein the active agent is a therapeutic agent selected from the group consisting of isotopes, drugs, toxins, fluorescent dyes activated by nonionizing radiation, hormones, hormone antagonists, receptor antagonists, enzymes or proenzymes activated by another agent, autocrine, and cytokine.
